(19) United States
(12) Patent Application Publication
(10) Pub. No.: US 2006/0122679 A1
(43) Pub. Date: Jun. 8, 2006

(54) Semiconductor-gated cardiac lead and method of use

(76) Inventors: Eric John Wengreen, Blaine, MN (US); Eric Falbe Hammill, Ham Lake, MN (US); Luke Thomas Babler, Shoreview, MN (US)

Correspondence Address: SCHWEGMAN, LUNDBERG, WOESSNER & KLUTH, 121 S. 8TH STREET, SUITE 1600, MINNEAPOLIS, MN, 55402, US

(21) Appl. No.: 11/004,547

(22) Filed: Dec. 3, 2004

(51) Int. Cl. A61N 1/05

(52) U.S. Cl. 607/122

(57) ABSTRACT

This document discusses, among other things a cardiac lead with first and second electrodes electrically interconnected by back-to-back diodes or another conductivity control device. During sensing of intrinsic electrical heart signals, these electrodes are isolated from each other by the conductivity control device. This reduces noise during sensing. During pacing or defibrillation, these electrodes are electrically connected to each other by the conductivity control device. When used in common as a return path electrode, this avoids unintentional stimulation of the heart at the return path electrode instead of at the stimulating electrode. The electrodes can also share a common conductor from one of the electrodes back to a proximal end of the lead.

### TECHNICAL FIELD

This patent document pertains generally to cardiac function management devices and more particularly, but not by way of limitation, to a semiconductor-gated cardiac lead and method of use.

### BACKGROUND

Implantable medical devices include, among other things, cardiac function management (CFM) devices such as pacers, cardioverters, defibrillators, cardiac resynchronization therapy (CRT) devices, as well as combination devices that provide more than one of these therapy modalities to a subject. Such devices often include one or more diagnostic capabilities. Moreover, such diagnostic capabilities may be used as a basis for automatically providing therapy to the subject or for communicating diagnostic information to a physician or to the subject.

One example of a diagnostic capability is sensing intrinsic electrical heart signals. These intrinsic heart signals include depolarizations that propagate through heart tissue. The depolarizations cause heart contractions for pumping blood through the circulatory system. The intrinsic heart signals are typically sensed by an implantable medical device at implanted electrodes. The implantable medical device typically includes sense amplifier circuits and other signal processing circuits to extract useful diagnostic information from the intrinsic heart signals.

Examples of therapeutic capability include delivering pacing-level stimulations intended to evoke responsive heart contractions. By appropriately timing the delivery of such stimulations, the patient's heart rate can be adjusted to help the heart provide adequate cardiac output. Moreover, regardless of whether heart rate is adjusted, the pacing-level stimulations can be used to spatially coordinate the heart depolarizations and associated heart contractions. This can also improve cardiac output. Cardiac function management devices can also provide antitachyarrhythmia pacing, cardioversion, or defibrillation shock therapy.

When a CFM device electronics unit is implanted in a patient, for example, pectorally or abdominally, an intravascular lead with one or more conductors is typically used to make one or more electrical connections between the implanted CFM device electronics unit and the heart. Such electrical connections permit sensing of intrinsic electric heart signals, delivery of pacing-level stimulations to evoke responsive heart contractions, or to deliver defibrillation shocks to interrupt certain tachyarrhythmias. In a typical construction, one conductor goes to each electrode at the distal portion of the lead. When multiple conductors are used, they typically run side by side and are insulated from each other and also from the patient's body. There is a need for improved intravascular cardiac leads, which should be small to avoid occluding the vasculature, and which should be strong enough to resist the considerable wear resulting from the continuous flexion resulting from heart contractions.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic drawing illustrating generally one example of a system that includes an implantable cardiac function management (CFM) device's electronics unit, which is coupled to a human or animal patient's heart by one or more electrodes located on distal portions of one or more intravascular or other leads.

FIG. 2 is a schematic illustration of one example of a distal portion of the lead, including the tip electrode, the ring electrode, the distal shock coil electrode, and the proximal shock coil electrode.

FIG. 3 is a schematic illustration of one example of a conductivity control device.

FIG. 4 is a cross-sectional diagram of at least some of the distal portion of the lead.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

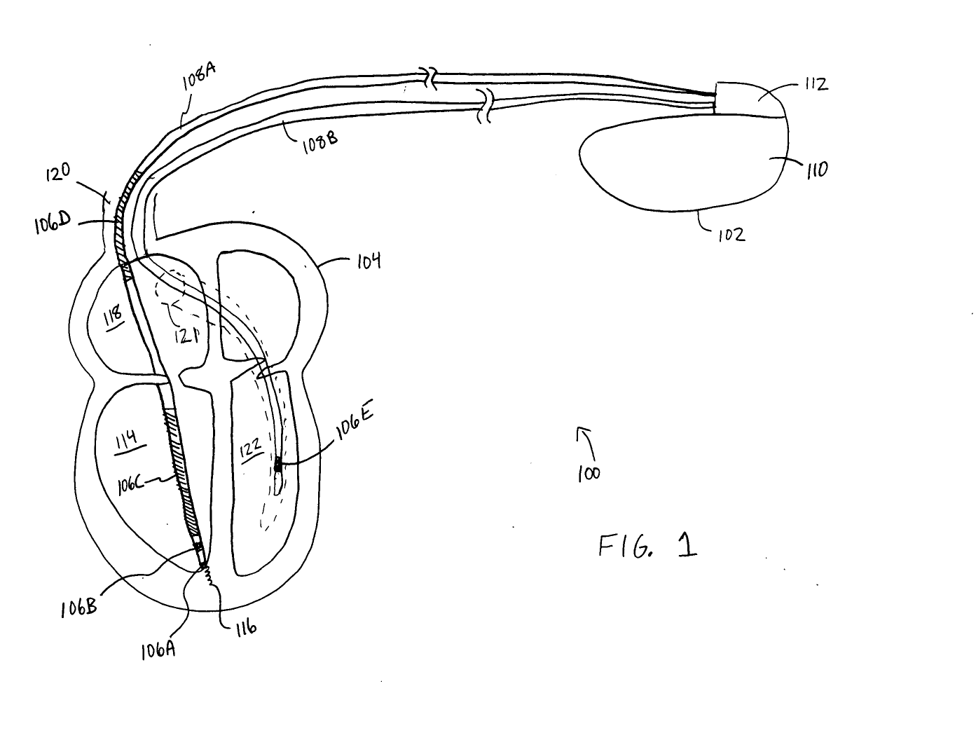

FIG. 1 is a schematic drawing illustrating generally one example of a system 100 that includes an implantable cardiac function management (CFM) device's electronics unit 102, which is coupled to a human or animal patient's heart 104 by one or more electrodes 106 located on distal portions of one or more intravascular or other leads 108. In this example, the CFM device electronics unit 102 is typically pectorally or abdominally implanted in the subject. The electronics unit 102 typically includes a hermitically sealed conductive housing 110 or “can,” from which an insulating header 112 extends. The insulating header 112 typically includes one or more receptacles for receiving a mating connector at a proximal end of a corresponding one of the leads 108. The housing 110 or the insulating header 112 can also be used to provide one or more additional electrodes for use in sensing intrinsic heart signals or sensing tissue impedance, or for delivering pacing or defibrillation therapy.

In the example of FIG. 1, the lead 108A extends intravascularly from the CFM device electronics unit 102 to right ventricle 114 of the heart 104. A distal end of the lead 108A, in this example, is affixed near an apex of the right ventricle 114 using a helical corkscrew-like or other affixation device 116. This helps to hold a distal “tip” pacing/sensing electrode 106A in contact with tissue of the right ventricular apex of the heart 104 for delivering pacing pulses or sensing intrinsic electrical heart signals. A “ring” or “band” pacing/sensing electrode 106B is typically located on the lead 108A in the right ventricle 114 at a location that is slightly proximal (e.g., about 12 millimeters) from the tip electrode 106A. The ring electrode 106B is typically used for delivering pacing pulses or sensing intrinsic electrical heart signals. A distal coil or other shock electrode 106C is typically located on the lead 108A in the right ventricle 114 at a location that is slightly proximal (e.g., either abutting or slightly separated) from the ring electrode 106B. The lead 108 also typically includes a proximal coil or other shock electrode 106D in a supraventricular location on the lead 108A, such as within the right atrium 118 or superior vena cava 120. The shock electrodes 106C-D are typically used to deliver a defibrillation shock to the heart 104, however, they may also be used to sense intrinsic electrical heart signals, in certain situations.

Each of the electrodes 106A-D is typically individually connected to the electronics unit 102 using its own conductor extending along and within the multiconductor lead 108A. These conductors are typically insulated from each other and the surrounding tissue, such that the electrodes 106A-D are typically only in electrical communication with each other through the heart tissue and body fluid of the patient's anatomy. Using fewer conductors reduces the diameter of the lead 108. This is highly desirable because the intravascular lead then occludes less of the vessel through which it extends or the heart chamber into which it is placed. Therefore, in one example, the “dedicated bipolar” lead configuration of FIG. 1 is replaced by an “integrated bipolar” lead configuration in which the ring electrode 106B abuts and electrically connects to the distal shock coil electrode 106C instead of being physically and electrically separated therefrom. This integrated bipolar configuration allows the ring electrode 106B and distal shock coil electrode 106C to share a common conductor that extends along the lead 108 back to the CFM device electronics unit 102.

When sensing intrinsic heart signals using the integrated bipolar configuration, however, the larger combined surface area of the commonly connected ring electrode 106B and distal shock electrode 106C may result in noisier sensed intrinsic heart signals. Such increased noise may result from increased sensing of myopotentials, electrical interference, or other interfering noise sources. However, the larger combined surface area of the commonly connected ring electrode 106B and distal shock electrode 106C may help reduce unintended anodal or other return electrode stimulation, such as when delivering a bipolar pacing-level stimulation pulse using the tip electrode 106A as a cathode for delivering the pacing pulse, and using as an anode the combined ring electrode 106B and distal shock electrode 106C. Such bipolar pacing pulses are typically intended to stimulate a responsive heart contraction at the cathodic tip electrode 106A. However, when the surface area of the return-path anode is too small, enough charge density can result at the anode to trigger an unintended anodic stimulation of the heart tissue. By connecting the distal shock electrode 106C in common with the anodic ring electrode 106B, a larger anodic surface area is present than when the ring electrode 106B is used alone as the anode. This reduces the areal anodic charge density, which, in turn, reduces the likelihood of producing an unintended anodic stimulation of the heart tissue.

Unintended anodic stimulation is even a bigger problem when the two electrodes between which a pacing pulse is delivered are farther apart. For example, when the tip electrode 106A is used as the pacing cathode and the nearby ring electrode 106B is used as the pacing anode, responsive heart contractions can typically be obtained at low pacing energy levels, such that the likelihood of an unintended anodic stimulation is reduced or avoided. However, left-sided pacing (e.g., biventricular pacing) and certain other configurations may require increased pacing energy levels. In one such example, the CFM device electronics unit 102 is coupled to an intravascular lead 108B that is threaded into the right atrium 118 through the superior vena cava 120, and then into the coronary sinus 121 and one of the veins extending into the heart wall from the coronary sinus 121. This allows one or more electrodes, such as ring electrode 106E, to be positioned within one of the coronary sinus veins in association with the intrinsic electrical heart signal conduction pathways of the left ventricle 122. The ring electrode 106E can be used as a cathode for delivering a pacing stimulation. Because of the larger distance and greater amount of myocardial tissue between the cathodic ring electrode 106E and the anodic ring electrode 106B (as compared with the distance between the cathodic tip electrode 106A and the anodic ring electrode 106B), a larger pacing energy is typically delivered using the anodic ring electrode 106E in order to evoke the desired left ventricular heart contraction. This larger energy may result in anodic stimulation at the ring electrode 106B, which could interfere with the desired spatial coordination of the left and right ventricular heart contractions. Accordingly, the present inventors have recognized that for avoiding such unintended anodic stimulation, it may be desirable to commonly connect the ring electrode 106B and the distal shock electrode 106C as a larger surface area anode return path for a pacing pulse, such as when the pacing pulse is delivered at a high enough energy, for example, from the left ventricular ring electrode 106E. However, the present inventors have also recognized that at times it may be desirable to isolate the distal defibrillation shock electrode 106C from the ring electrode 106B, such as when ring electrode 106B is being used in conjunction with another electrode for bipolar sensing (e.g., between two relatively closely spaced electrodes) or unipolar sensing (e.g., sensing between two more distantly spaced electrodes) of intrinsic electrical heart signals. Further still, the present inventors have recognized that there may be opportunities for the ring electrode 106B and the distal shock coil electrode 106C to share a substantial portion of a common conductor back through the lead to the CFM device electronics unit 102, thereby allowing the diameter of the lead 108A to be reduced as compared to when individual conductors are provided.

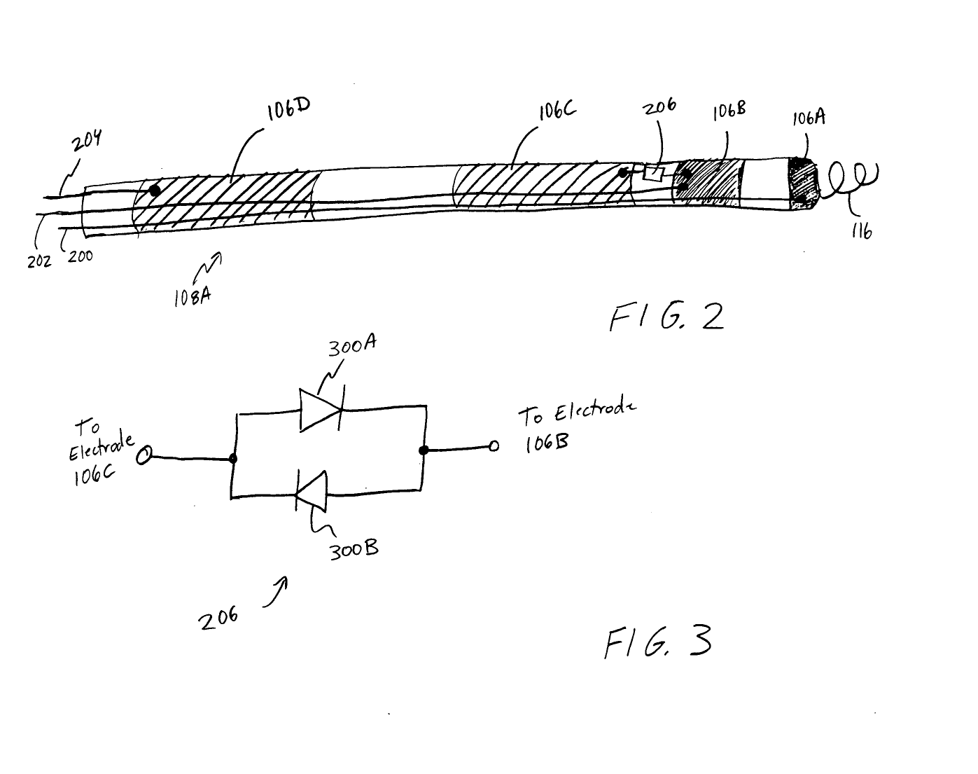

FIG. 2 is a schematic illustration of one example of a distal portion of the lead 108A, including the tip electrode 106A, the ring electrode 106B, the distal shock coil electrode 106C, and the proximal shock coil electrode 106D. In the example of FIG. 2, the tip electrode 106A, the ring electrode 106B, and the proximal shock coil electrode 106D are individually connected to respective couplings on a connector to the CFM device electronics unit 102 at a proximal end of the lead 108A by corresponding individual conductors 200, 202, and 204. In this example, the distal shock coil electrode 106C does not have its own individual conductor back along the lead 108A to the CFM device electronics unit 102. Instead, in this example, the distal shock coil electrode 106C is electrically connected to the ring electrode 106B through a conductivity control device 206, such as the back-to-back (e.g., antiparallel) semiconductor diodes 300A-B illustrated in FIG. 3. In this manner, the distal shock coil electrode 106C is connected back to the CFM device electronics unit 102 through the conductivity control device 206 and the conductor 202 that is shared between the ring electrode 106B and the distal shock coil electrode 106C.

FIG. 3 is a schematic illustration of one example of a conductivity control device 206. In this example, the conductivity control device 206 includes back-to-back (e.g., antiparallel) semiconductor rectifier diodes 300A-B. In this example, the diode 300A has its anode connected to the distal shock electrode 106C and its cathode connected to the ring electrode 106B, and diode 300B has its anode connected to the ring electrode 106B and its cathode connected to the distal shock coil electrode 106C. In one example, the diodes 300A-B are customized diodes, which are available commercially from Microsemi Corp. of Irvine, Calif., and which, in one example, provide a threshold voltage of less than about 1.5 Volts and a current handling of 70 amperes for 3 milliseconds or 10 amperes for 30 milliseconds.

In this example, the diodes 300A-B each have a turn-on threshold voltage that exceeds a voltage amplitude level associated with a sensed intrinsic heart depolarization. However, in this example the turn-on threshold voltage is less than a voltage amplitude level associated with a pacing-level stimulation to be delivered to evoke a responsive heart contraction. For example, typical cardiac depolarization amplitudes (e.g., QRS amplitudes associated with a ventricular depolarization and ventricular heart contraction, or P-wave amplitudes associated with an atrial depolarization and atrial heart contraction) are in the range of between about 5 millivolts and about 20 millivolts. Because of the selection of the turn-on threshold voltage value of the conductivity control device 206, the conductivity control device 206 will not conduct even when the full voltage drop of an intrinsic heart depolarization (e.g., 5-20 mV) appears between the ring electrode 106B and the distal shock coil electrode 106C. Therefore, for sensing intrinsic electrical heart depolarizations, the distal shock coil electrode 106C is electrically isolated from the ring electrode 106B by the conductivity control device 206. This avoids introducing additional far-field noise during such sensing from the additional surface area of the distal shock coil electrode 106C.

Because the turn-on threshold voltage is less than a voltage amplitude level associated with a pacing-level stimulation, the conductivity control device 206 turns on and electrically conducts between the ring electrode 106B and the distal shock coil electrode 106C during delivery of a pacing pulse. This allows the ring electrode 106B and the distal shock coil electrode 106C to be used in common as a combined larger surface area anode return path for a pacing pulse being delivered by another electrode, such as the left ventricular ring electrode 106E. This larger combined surface area anode will have a smaller charge density than the ring electrode 106B used alone, therefore anodal stimulation is much less likely to occur when the conductivity control device 206 is turned on to conduct. Because pacing voltages are typically greater than 1.0 Volts, a turn-on threshold voltage of less than 1.0 Volts can be used. In one example, the turn-on threshold voltage is between 0.5 Volts and 0.7 Volts, as is provided by using semiconductor rectification diodes 300A-B.

In an example such as that of FIG. 3, in which a defibrillation shock is to be provided, the diodes 300A-B should be capable of handling the high current levels associated with the defibrillation shock. However, Schottky diodes having a turn-on threshold voltage of about 0.3 Volts could also be used if they could be fabricated in such a manner as to handle the defibrillation shock currents, such as where the CFM device electronics unit is programmable to deliver even lower pacing voltages, such as pacing voltages of about 0.5 Volts. However, this is not required, because at such low pacing voltages, the risk of unintended anodal stimulation is lower, therefore, the surface area of ring electrode 106B alone will typically be adequate for such lower pacing voltage. In one example, the turn-on threshold voltage of the conductivity control device 206 is between about 20 millivolts and about 10 Volts. In another example, the turn-on threshold voltage is between about 300 millivolts and about 1.0 Volts. In yet another example, the turn-on threshold voltage is between 400 millivolts and 0.8 Volts. In yet a further example, the turn-on threshold voltage is about 0.7 Volts. Moreover, the present systems and methods are also useful for a pacing lead that does not deliver a defibrillation shock, in which case the diodes 300A-B need not be capable of handling the high current levels associated with the defibrillation shock.

In the above example, the defibrillation shock coil electrodes 106C-D will also typically be used to deliver a monophasic or biphasic defibrillation shock therebetween. The defibrillation shock typically involves energies of up to about 35 Joules at voltages of up to about 780 Volts. During such a defibrillation shock, the conductivity control device 206 will turn on to provide a conduction path from the distal shock coil electrode 106C back through the ring electrode 106B and the commonly shared conductor 202 back to the CFM device electronics unit 102. The back-to-back diode configuration of FIG. 3 ensures that such a conduction path will be available regardless of the defibrillation shock voltage polarity, because the voltage polarity will switch during a biphasic defibrillation shock pulse. Each of the diodes 300A-B should be capable of carrying up to 70 amperes of current during such a defibrillation shock.

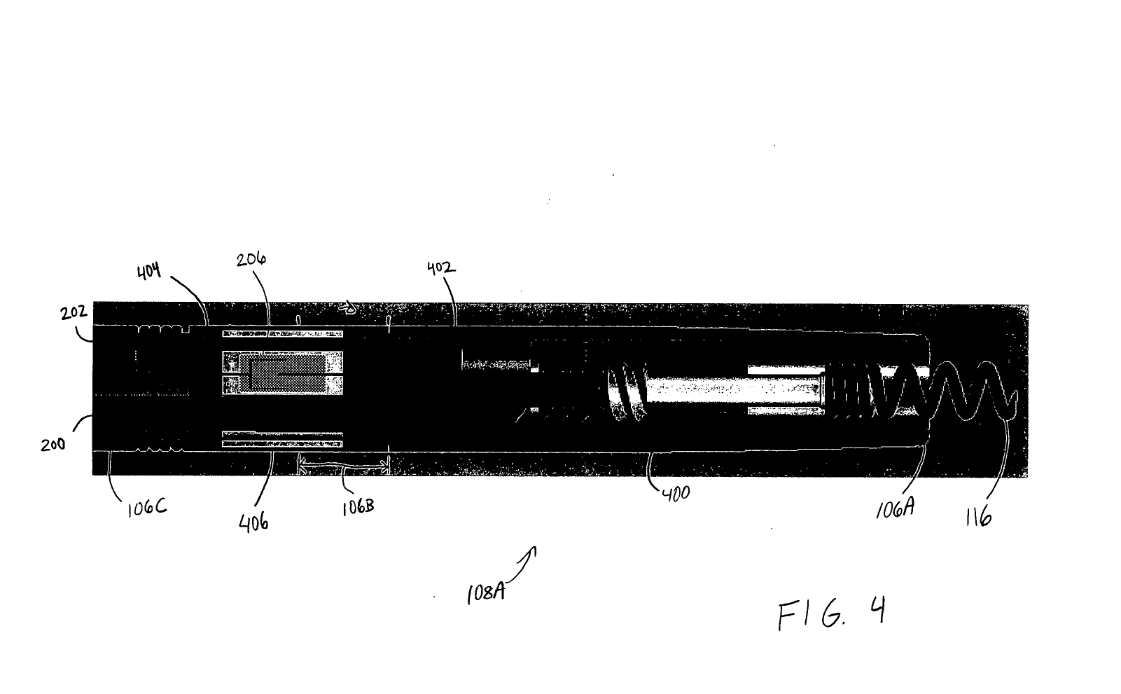

FIG. 4 is a cross-sectional diagram of at least some of the distal portion of the lead 108A. In this example, the tip electrode 106A is exposed only at the distal end of the lead 108A, and is separated from the exposed portion of the ring electrode 106B by a silicone, polyurethane, or other insulating portion 400. In this example, the conductor 200 electrically connecting the distal tip electrode 106A to the proximal end of the lead 108A and the CFM device electronics unit 102 can be rotated at the proximal end of the lead 108A. The resulting torsion of the conductor 200 at its threaded connection to an internal portion of the distal tip electrode 106A at the distal end of the lead 108A extends or retracts the helical or other tip affixation device 116 from within an interior cavity of the distal tip electrode 106A.

In the example of FIG. 4, the conductor 202 electrically connects to an internal block portion 402 of the ring electrode 106B. The conductor 200 passes through (and is insulated from) a lumen in the block portion 402 of the ring electrode 106B. The internal block portion 402 of the ring electrode 106B is connected through the conductivity control device 206 to an internal end block portion 404 of the distal shock coil electrode 106C. The internal block portion 402 of the ring electrode 106B and the end block portion 404 of the distal shock coil electrode 106C are otherwise separated by an insulator 406 which completes a hermetically sealed cavity in which the conductivity control device 206 is disposed.

Although the above example has emphasized using a conductivity control device 206 between a distal shock coil electrode 106C and a slightly more distal ring electrode 106B, this is merely a particularly useful example (for the reasons discussed above) that is presented to assist the reader's conceptualization. However, the conductivity control device 206 could be inserted electrically between any two cardiac electrodes to allow use of both electrodes during pacing and defibrillation, while isolating the two electrodes from each other during sensing of intrinsic electrical heart signals, as discussed above. This can be used to reduce far-field noise during sensing and to avoid unintentional stimulation at a return path electrode during pacing or resynchronization therapy when pacing-level energy stimulations are delivered.

Moreover, in the above example, if a polarity of the pacing voltage received at the distal shock coil electrode 106C is known with respect to the tip electrode 106A, then a single diode could be used in place of the back-to-back diodes shown in FIG. 3. However, in such a case, the polarity of the pacing voltage received at the distal shock coil electrode 106C must be the same polarity as any defibrillation shock voltage present at the distal shock coil electrode 106C, since the unidirectional diode should conduct both during the pacing-level pulse and the defibrillation shock level pulse. Therefore, such a single diode configuration should be avoided where a biphasic defibrillation pulse (which switches polarity) is used.

Furthermore, although the above description has emphasized avoiding unwanted anodal stimulation at the return electrode, the description is presented this way merely to assist the user's conceptualization, since the tip electrode 106A is typically used as a cathode. However, the present techniques are equally applicable where the return electrode is used as a cathode in conjunction with another electrode being used as an anode. In certain such situations, it is still desirable to avoid unintentional stimulation at the return path electrode (which, in this case, is now a cathode, making such unwanted return path electrode stimulation an unintentional cathodic stimulation).

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. An apparatus comprising:
an elongate cardiac lead body, sized and shaped to permit introduction into a human or animal subject, the lead body including a proximal portion and a distal portion;
at least one electrical coupling, near the proximal portion of the lead body, to permit an electrical connection to an implantable electronics unit;
a first electrode, at the distal portion of the lead body;
a second electrode, at the distal portion of the lead body;
a conductivity control device, at the distal portion of the body, the conductivity control device electrically between the first and second electrodes, the conductivity control device including a turn-on characteristic that allows substantial current conduction between the second electrode and the electronics unit only when a voltage at the first or second electrode exceeds a turn-on threshold voltage value;
wherein the turn-on threshold voltage value exceeds a voltage amplitude level associated with a sensed intrinsic heart depolarization; and
wherein the turn-on threshold voltage value is less than a voltage amplitude level associated with a pacing stimulation to be delivered to evoke a responsive heart contraction.

2. The apparatus of claim 1, in which:
the first electrode is located slightly proximal from a distal end of the distal portion of the lead body; and
the second electrode is a shock electrode located proximal from the first electrode, and in which the first and second electrodes are electrically isolated from each other except through the conductivity control device and through nearby tissue.

3. The apparatus of claim 2, in which the first electrode is a ring electrode, and in which the apparatus further includes a third electrode at the distal end of the distal portion of the lead body.

4. The apparatus of claim 1, further comprising an electrical conductor extending along the elongate lead body from the second electrode to the at least one electrical coupling near the proximal portion of the lead body, the conductor shared by the first and second electrodes in that the conductor electrically connects the first electrode to the at least one electrical coupling, and the conductor also electrically connects the second electrode to the at least one electrical coupling through the conductivity control device.

5. The apparatus of claim 1, in which the conductivity control device includes a semiconductor diode.

6. The apparatus of claim 5, in which the conductivity control device includes back-to-back semiconductor diodes, the back-to-back semiconductor diodes including first and second diodes, the first diode having an anode electrically connected to the first electrode and a cathode electrically connected to the second electrode, the second diode having an anode electrically connected to the second electrode and a cathode electrically connected to the first electrode.

7. The apparatus of claim 1, in which the turn-on threshold voltage is between 20 millivolts and 10 Volts.

8. The apparatus of claim 7, in which the turn-on threshold voltage is between 300 millivolts and 1.0 Volts.

9. The apparatus of claim 8, in which the turn-on threshold voltage is between 400 millivolts and 0.8 Volts.

10. The apparatus of claim 9, in which the turn-on threshold voltage is about 0.7 Volts.

11. The apparatus of claim 1, further comprising a cardiac function management device connected to the lead body.

12. The apparatus of claim 11, further comprising an external interface device communicatively coupled to the cardiac function management device.

13. A method comprising:
using first, second, and third electrodes located in electrical association with a heart; and
controlling an electrical conductivity between the first and second electrodes to electrically conduct between the first and second electrodes when a pacing-level stimulation is delivered using the third electrode, and to electrically isolate between the first and second electrodes when an intrinsic depolarization level voltage is present at the second electrode.

14. The method of claim 13, further comprising using a commonly shared conductor that is shared by the first and second electrodes from an implantable cardiac function management device connector to the first electrode.

15. The method of claim 13, further comprising:
delivering a pacing-level simulation using the first, second, and third electrodes; and
providing a current path during the delivered pacing-level stimulation, the current path allowing conduction from each of the first and second electrodes to the implantable cardiac function management device electronics unit.

16. The method of claim 15, in which the delivering the pacing-level stimulation at the third electrode includes using the third electrode as a cathode and delivering the pacing-level stimulation at an energy level that would cause anodal stimulation of the heart by the first electrode if the first electrode was electrically isolated from the second electrode.

17. The method of claim 15, in which the delivering the pacing-level stimulation at the third electrode includes using the third electrode as a cathode and delivering the pacing-level stimulation at an energy level that would risk anodal stimulation of the heart by the first electrode if the first electrode was electrically isolated from the second electrode.

18. The method of claim 15, further comprising:
sensing an intrinsic heart depolarization using the first electrode; and
isolating the second electrode from the first electrode during the sensing of the intrinsic heart depolarization.

19. The method of claim 18, further comprising:
delivering a defibrillation shock using the second electrode; and
electrically conducting between the first and second electrodes during the delivering the defibrillation shock.

20. The method of claim 13, in which the using first, second, and third electrodes located in electrical association with a heart includes:
using a first electrode that is a ring electrode located in a right ventricle of the heart;
using a second electrode that is a shock electrode substantially located in a right ventricle of the heart; and
using a third electrode that is a pacing electrode located in a coronary sinus vein in association with a left ventricle of the heart.

21. The method of claim 13, in which the using first, second, and third electrodes located in electrical association with a heart includes using at least one electrode in association with a left ventricle of the heart.

22. The method of claim 13, in which the using first, second, and third electrodes located in electrical association with a heart includes using at least one epicardial electrode.

23. A system comprising:
a first elongate lead body, sized and shaped to permit introduction into a human or animal subject, the first lead body including a proximal portion and a distal portion;
a first electrode, configured as a ring pacing electrode at the distal portion of the lead body;
a second electrode, configured as a coil shocking electrode at the distal portion of the lead body;
a third electrode, configured as a tip pacing electrode at a distal end of the distal portion of the lead body;
antiparallel diodes, at the distal portion of the body, the conductivity diodes electrically between the first and second electrodes, the diodes including a turn-on characteristic that allows substantial current conduction between the second electrode and the electronics unit only when a voltage at the first or second electrode exceeds a turn-on threshold voltage value;
wherein the turn-on threshold voltage value exceeds a voltage amplitude level associated with a sensed intrinsic heart depolarization; and
wherein the turn-on threshold voltage value is less than a voltage amplitude level associated with a pacing stimulation to be delivered to evoke a responsive heart contraction.

24. The system of claim 23, further comprising a second elongate lead body, sized and shaped to permit introduction into a human or animal subject, the second lead body including a proximal portion and a distal portion, the distal portion including a fourth electrode, sized and shaped to be located in a coronary sinus vein in association with a left ventricle.

25. The system of claim 23, further comprising a cardiac function management device connected to the lead body, the cardiac function management device programmed to deliver a pacing stimulation between (1) the fourth electrode, and (2) the first and second electrodes in common, and wherein an energy level of the pacing stimulation would induce anodal stimulation at the first electrode if the first electrode were not commonly connected to the second electrode, but wherein anodal stimulation is avoided by the additional surface area of the second electrode by its common connection to the first electrode during the delivery of the pacing stimulation.

26. An apparatus comprising:
an elongate cardiac lead body, sized and shaped to permit intravascular introduction into a human or animal subject, the lead body including a proximal portion and a distal portion;
at least one electrical coupling, near the proximal portion of the lead body, to permit an electrical connection to an implantable electronics unit;
a first electrode, at the distal portion of the lead body;
a second electrode, at the distal portion of the lead body;
a conductivity control device, at the distal portion of the body, the conductivity control device electrically between the first and second electrodes, the conductivity control device including a turn-on characteristic that allows substantial current conduction between the second electrode and the electronics unit only when a voltage at the first or second electrode exceeds a turn-on threshold voltage value that is between about 0.5 Volts and 0.8 Volts.
